(19) United States
(12) Patent Application Publication
(10) Pub. No.: US 2011/0106121 A1
(43) Pub. Date: May 5, 2011

(54) Hand-held ophthalmic polishing instrument

(76) Inventor: Roberto Zaldivar, Ciudad (AR)

(21) Appl. No.: 12/589,898

(22) Filed: Oct. 30, 2009

(51) Int. Cl. A61F 9/007

(52) U.S. Cl. 606/162

(57) ABSTRACT

A surgical instrument and method for polishing or cleaning epithelial cells which reside in the posterior capsule of the eye is provided. The instrument is configured to have a flexible foam tip which is applied to the cells at an angle. This allows the instrument to fully access the posterior capsule and facilitates complete polishing or cleaning of the lens of the eye.

### BACKGROUND OF THE INVENTION

1. Field of the Invention

The present invention generally relates to surgical instruments and more specifically to a hand-held instrument particularly useful in ophthalmological procedures. The invention is applicable to the polishing or cleaning of the capsule of the lens.

2. Description of the Related Art

The human eye includes a lens enclosed by a transparent capsule. Cataract is a condition characterized by opacity of the lens causing partial or total blindness. Cataract surgery is a common surgical procedure during which a patient's cloudy natural lens is removed and replaced with a synthetic lens or intraocular lens to restore the lens's transparency. The natural lens of the eye is situated in an elastic-like capsular bag that holds it in place. The front portion of the capsule is opened up during surgery. After removing the natural lens, the intraocular lens is positioned inside the capsular bag. Generally, the intraocular lens is capable of improving the transmission of light, and reducing the scattering, the absorption or both. Although this surgery is successfully performed on millions of people in United States alone each year, there are numerous complications that may arise after the surgery.

Posterior capsular fibrosis or secondary opacification is a late post-operative event which may develop in cataract surgery patients. Patients report such symptoms such as decreased vision, photosensitivity and glare which are caused by proliferation of equatorial lens epithelium along the posterior capsule of the lens. This proliferation thickens and clouds the posterior capsule.

Currently, there are several ways to combat fibrosis or opacification of the posterior capsule. One approach is to design the intraocular lens with various shapes and materials. For instance, it has been clinically shown that lens with square edges present a barrier to proliferation of epithelial cells that cause secondary opacity (Nishi et al, 1996). One drawback of this approach is that although an intraocular lens with square edges improves central capsular opacity, the edges of the lens cause distortions to light and diminish the optical quality.

A second approach involves using chemical and pharmacologic ways to prevent lens capsule opacification (U.S. Pat. No. 4,515,794). For example, chemical and radiation means have been attempted to lower the incident of opacification after cataract growth (Roy et al., 1979). However, side effects from the inhibitory compounds were found to have deletory effects to the cornea and iris, making them unsafe. For the most part, chemical and radiation methods have not gained wide acceptance.

Another approach is the use of handpiece systems which can be used during capsulotomy (U.S. Pat. No. 5,921,999). These instruments can depolish or rough an area of interest and may also be attached to a vacuum which aspire portions of the epithelial cells in the capsular bag. The downside of these instruments is that while they have easy access to the posterior side of the sack and some portions of the anterior side, a person having ordinary skill in this art has difficulty accessing the equatorial zone. As a result, these instruments fail to clean all the epithelial cells of the capsular bag. Furthermore, because of their coarseness, the instruments can break or tear thin fibers that surround the region.

Furthermore, most of these approaches are expensive and require time to carry out.

Thus, the prior art is deficient in an instrument that is designed to allow access to the equatorial zone in order to quickly and completely clean the epithelial cells of the capsular bag without damaging it. The present invention fulfills this long-standing need and desire in the art.

### SUMMARY OF THE INVENTION

In view of the disadvantages inherent in the known types of ophthalmic surgical systems and instruments now present and available in the prior art, the present invention provides a surgical instrument that is inexpensive to manufacture, flexible to use and allows complete polishing of epithelium cells that reside in the capsular bag of the eye.

In one embodiment of the present invention, there is provided ophthalmic surgical instrument for polishing the capsule of an eye comprising: a body having a top end and a bottom end, wherein the length of the body is at least two times longer than the thickness of the body; a tapered portion connected to the top end of the body, wherein said tapered portion is integrally joined to the body at an acute angle of from about 0.1 degrees to about 10 degrees; and a biocompatible swab having a diameter from about 0.1 mm to about 5 mm attached to the tip of the tapered portion.

In another embodiment of the present invention, there is provided an ophthalmic surgical instrument for polishing the capsule of an eye comprising: a ductile body having a top end and a bottom end, wherein the length of said body is at least five times longer than the thickness of the body; a tapered portion connected to the top end of the body, wherein the tapered portion is integrally joined to the body at an acute angle between about 0.1 degrees to about 25 degrees; and a biocompatible swab having a diameter between about 0.1 mm to about 5 mm attached to the tip of the tapered portion.

The present invention is also directed to a method of treating posterior capsular fibrosis or secondary opacification in an individual, comprising polishing epithelium cells from the capsule of an eye using a surgical instrument described herein.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 shows the surgical instrument and its typical dimensions.

FIG. 2 shows a close-up views of the flexible foam tip.

FIG. 3 depicts a cross section view of the human eye.

FIG. 4 depicts a cross section view of the human lens.

FIG. 5 shows the human lens after cataract removal. It also shows the epithelial cells which reside in the capsular bag.

FIG. 6 shows the surgical instrument polishing epithelial cells which reside in the posterior capsule.

FIG. 7 shows the lens of the human eye after epithelial cells have been completely polished.

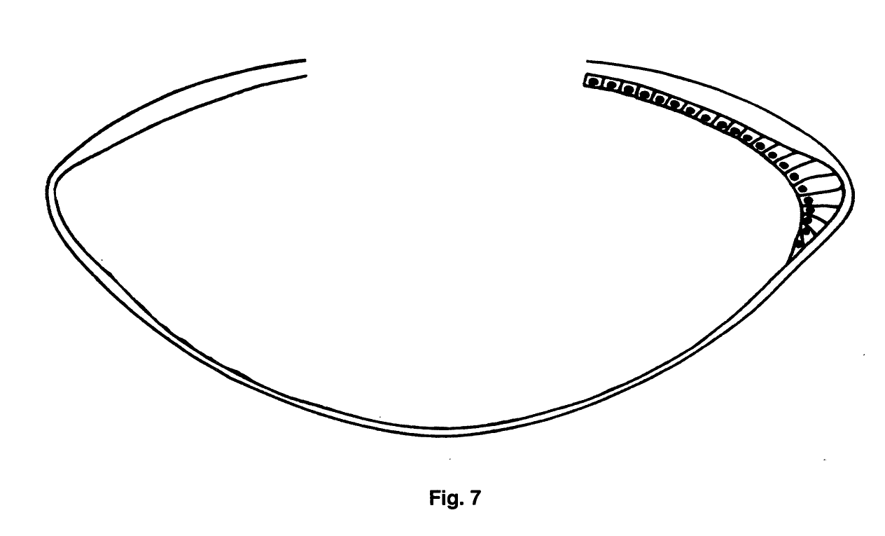

### DETAILED DESCRIPTION OF THE INVENTION

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

The present invention is directed to an ophthalmic surgical instrument for polishing the capsule of an eye comprising a body having a top end and a bottom end, wherein the length of said body is at least two times longer than the thickness of said body; a tapered portion connected to the top end of the body, wherein said tapered portion is integrally joined to said body at an acute angle of from about 0.1 degrees to about 25 degrees; and a biocompatible swab having a diameter from about 0.1 mm to about 5 mm attached to the tip of said tapered portion. Preferably, the body of the instrument of the present invention may be composed of a material which is flexible yet stiff as is known to those of ordinary skill in this art. For example, representative materials for the body of the instrument of the present invention are selected from the group consisting of synthetic polymers, polycarbonate, polypropylene, polyethylene, polyurethane, plastics, metals and any other suitable industrial material as would be well known to one having ordinary skill in this art.

Preferably, the biocompatible swab of the instrument of the present invention may be composed of a material which is delicate yet abrasive as is known to those of ordinary skill in this art. For example, representative materials for the biocompatible swab of the instrument of the present invention include but are not limited to those selected from the group consisting of nylon, silicone, acrylic compounds, teflon, polyesters, polyethylene, polyurethane, plastic and any other suitable industrial material as would be well known to one having ordinary skill in this art. The biocompatible swab of the instrument of the present invention may have any shape or form necessary to facilitate the function contemplated herein. For example, the biocompatible swab may have a spherical diffuse shape. A person having ordinary skill in this art would readily recognize that one may clean the cells using mechanical polishing or by applying a liquid cleaning substance in conjunction with use of the instrument of the present invention.

The total length of the instrument may be varied but preferably is between about 2 cm to about 18 cm and the length of the tapered portion is between about 0.1 cm to about 3 cm. Similarly, the weight of the instrument is between 1 grams and 100 grams or heavier as desired. In one embodiment, the body is cylindrically shaped but may also be any other suitable shape as would be well known to one having ordinary skill in this art, including but not limited to, triangular shapes or rectangular shapes.

The present invention is further directed to an ophthalmic surgical instrument for polishing the capsule of an eye comprising: a ductile body having a top end and a bottom end, wherein the length of said body is at least two times longer than the thickness of said body; a tapered portion connected to the top end of the body, wherein said tapered portion is integrally joined to said body at an acute angle between about 0.1 degrees to about 10 degrees; and a biocompatible swab having a diameter between about 0.1 mm to about 5 mm attached to the tip of the tapered portion. Preferably, the Young's modulus of said body and said tapered portion is between about 0.1 GPa to about 15 GPa. Preferably, the body of the instrument of the present invention may be composed of a material which is flexible yet stiff as is known to those of ordinary skill in this art. For example, representative materials for the body of the instrument of the present invention are selected from the group consisting of synthetic polymers, polycarbonate, polypropylene, polyethylene, polyurethane. Preferably, the biocompatible swab of the instrument of the present invention may be composed of a material which is delicate yet abrasive as is known to those of ordinary skill in this art. For example, representative materials for the biocompatible swab of the instrument of the present invention include but are not limited to those selected from the group consisting of nylon, silicone, acrylic compounds, teflon, polyesters, polyethylene, polyurethane, plastics, metals and any other suitable industrial material as would be well known to one having ordinary skill in this art. The biocompatible swab of the instrument of the present invention may have any shape or form necessary to facilitate the function contemplated herein. For example, the biocompatible swab may have a spherical diffuse shape. The total length of the instrument (l₁) may be varied but preferably is between about 2 cm to about 18 cm and the length of the tapered portion (l₂) is between about 0.1 cm to about 3 cm. Similarly, the weight of the instrument is between 1 grams and 100 grams or heavier as desired. In one embodiment, the body is cylindrically shaped but may also be any other suitable shape as would be well known to one having ordinary skill in this art, including but not limited to, triangular shapes or rectangular shapes.

The present invention is further directed to a method of preventing posterior capsular fibrosis or secondary opacification in an individual, comprising: polishing epithelium cells from the capsule of an eye using an ophthalmic surgical instrument for polishing the capsule of an eye comprising a body having a top end and a bottom end, wherein the length of said body is at least two times longer than the thickness of said body; a tapered portion connected to the top end of the body, wherein said tapered portion is integrally joined to said body at an acute angle of from about 0.1 degrees to about 25 degrees; and a biocompatible swab having a diameter (l₃) from about 0.1 mm to about 5 mm attached to the tip of said tapered portion. In one aspect of the methods of the present invention, the method may be utilized to clean epithelial cells and cortical residual material so as to help prevent secondary opacification and posterior capsular fibrosis. In another aspect of this method of the present invention, the method may be utilized to directed to clean cortical residual material only. In yet another embodiment, this method may further comprise applying a liquid to said cells. For example, as would be readily recognizable to a person having ordinary skill in this art, one may apply said liquid to clean said cells, to kill said cells or to inhibit the migration of said cells. Representative liquids for these purposes, such as saline or various alcohols are well known in the art.

The present invention is further directed to a method of preventing posterior capsular fibrosis or secondary opacification in an individual, comprising: polishing epithelium cells from the capsule of an eye using an ophthalmic surgical instrument for polishing the capsule of an eye comprising: a ductile body having a top end and a bottom end, wherein the length of said body is at least two times longer than the thickness of said body; a tapered portion connected to the top end of the body, wherein said tapered portion is integrally joined to said body at an acute angle between about 0.1 degrees to about 10 degrees; and a biocompatible swab having a diameter between about 0.1 mm to about 5 mm attached to the tip of said tapered portion. In yet another embodiment, this method may further comprise applying a liquid to said cells. For example, as would be readily recognizable to a person having ordinary skill in this art, one may apply said liquid to clean said cells, to kill said cells or to inhibit the migration of said cells. Representative liquids for these purposes, such as saline or various alcohols are well known in the art.

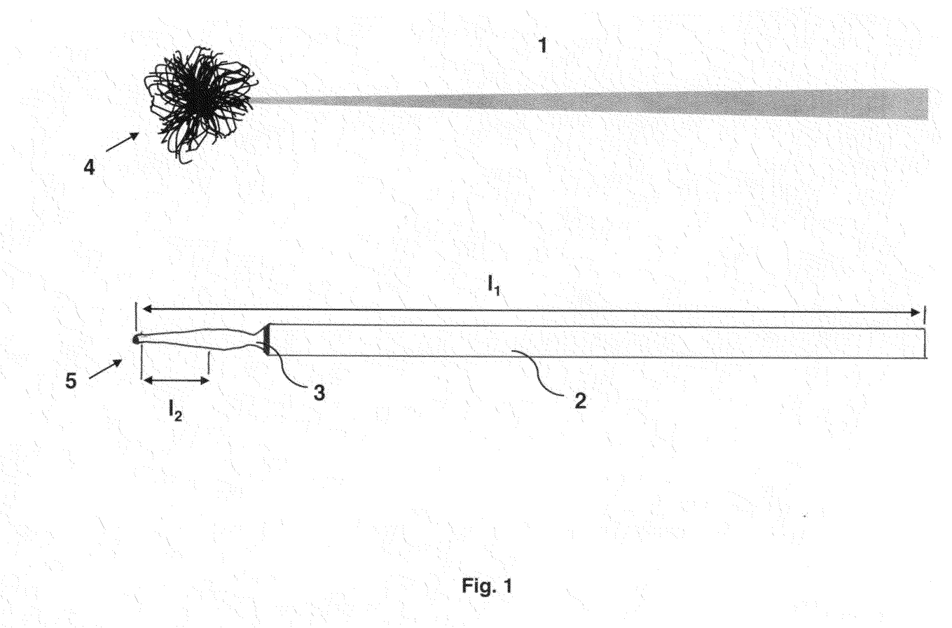

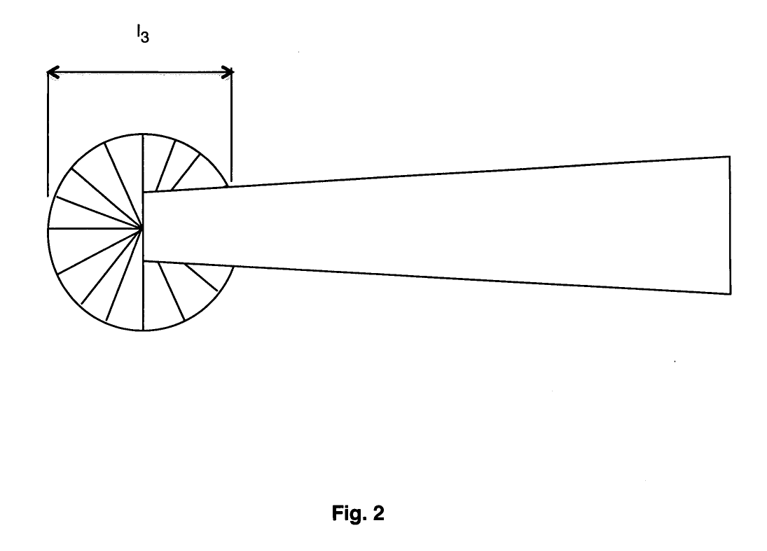

The instruments of the present invention may be further described and understood with reference to the Figures. For example, FIG. 1 provides an ophthalmic surgical instrument, generally designated 1, which includes the body 2, a bent neck 3 and a soft swab 4 attached to the head 5. One embodiment of the invention is shown in FIG. 1 along with the dimensions of the instrument. As shown, the neck of the instrument tapers to the head where the flexible foam is attached. FIG. 1 also shows the dimensional ratios of the body, neck and tip. FIG. 2 shows a close-up schematic view of the head.

In a presently preferred embodiment, the outside of body 2 of the instrument is made of plastic such as polyethylene or polyurethane. In the presently preferred embodiment, the foam 4 is made of nylon, silicone, acrylic compounds, teflon, polyesters, polyethylene, polyurethane, plastics, metals or the like.

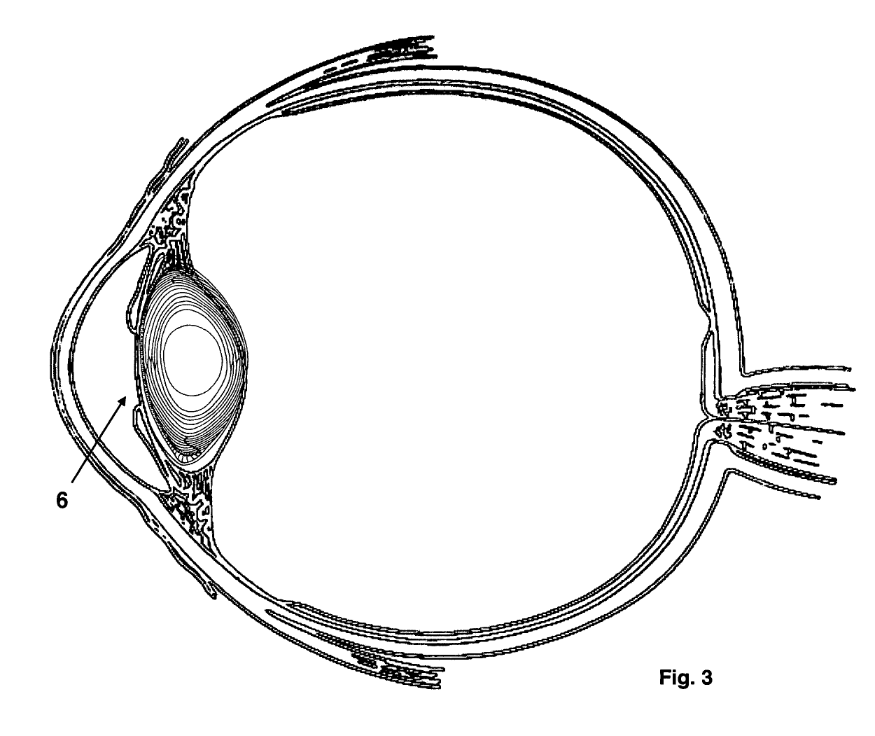

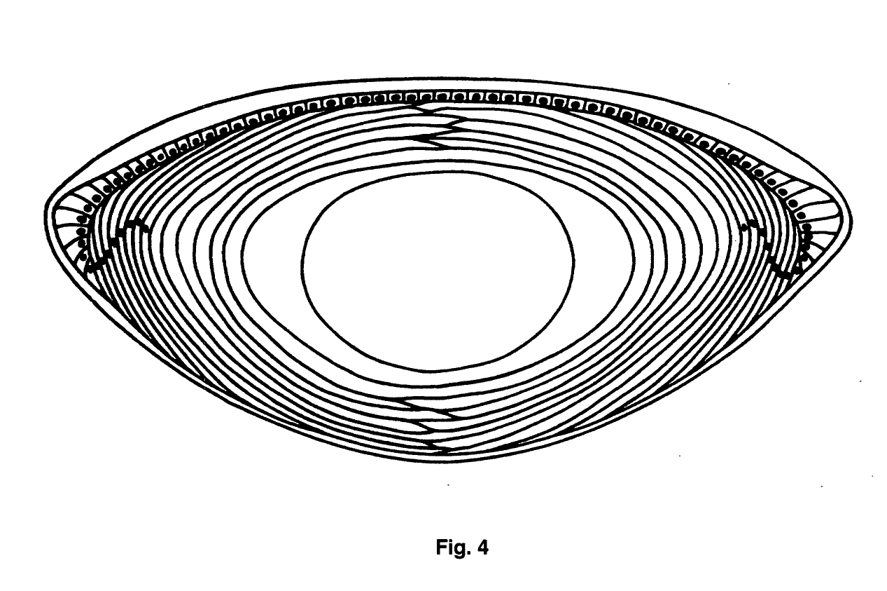

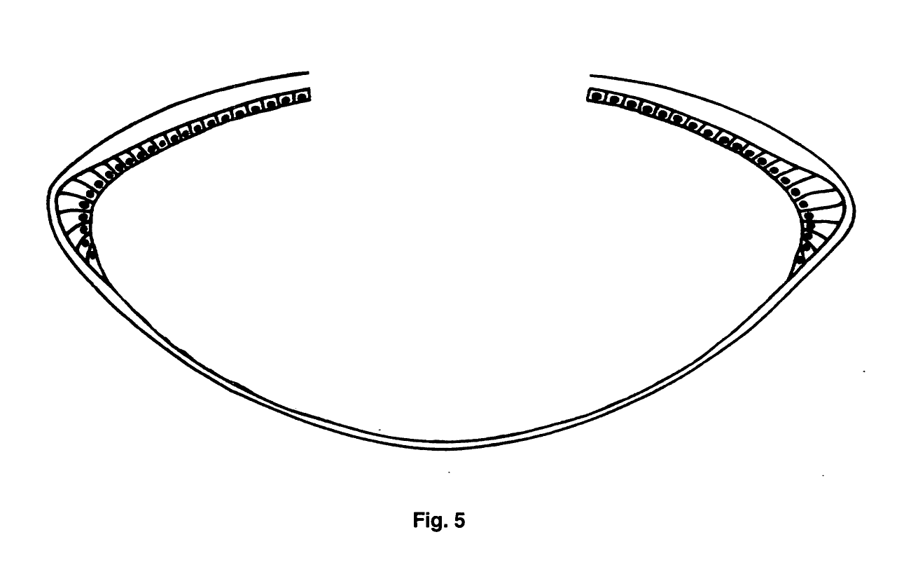

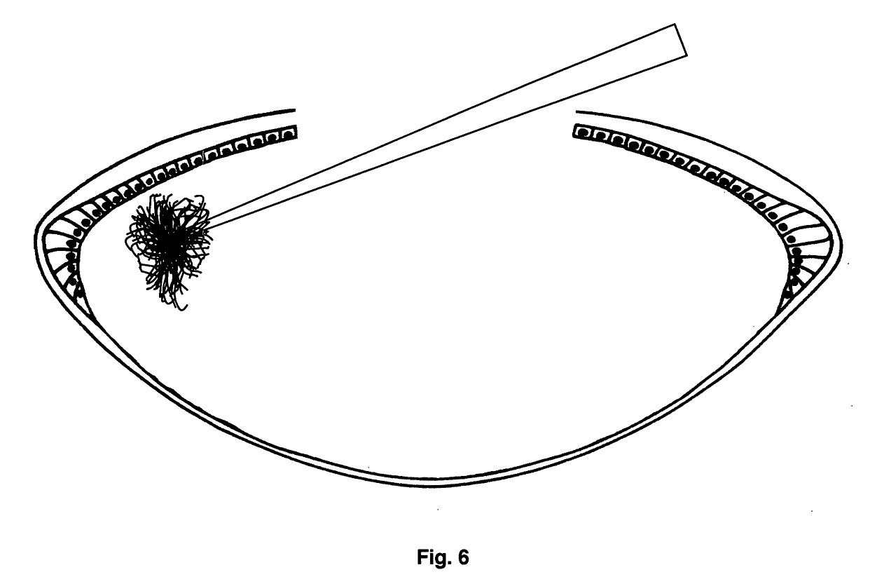

In a presently preferred embodiment, the instrument of FIGS. 1 and 2 is used to treat and/or clean an individual's lens 6 (FIGS. 3-4). Individuals needing such a treatment may be suffering from posterior capsule fibrosis or secondary opacification which arise from proliferation of equatorial lens epithelium (FIG. 5) along the posterior capsule of the lens. The shape and material of the surgical instrument allows for easy access of the epithelial cells which resides in the capsular bag. The hand application of the instrument polishes or cleans the epithelial cells (FIG. 6) resulting in a cleared lens without the epithelium.

The bent angled neck allows the instrument to access the equatorial zone of the capsule which is problematic for other instruments. The angle can range from about 5 degrees to about 30 degrees. The ductile material ensures that damaging pressure is not applied to the eye. The key advantage of the present invention is that it may be manufactured at low cost, making it disposable, and provides quick access to epithelial cells residing in the capsular region without damaging the eye. Since the instrument may be used manually by hand, no calibration is needed.

While presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

### REFERENCES

“Explanation of Endocapsule Posterior Chamber Lens After Spontaneous Posterior Dislocation”, Nishi et al, J Cataract & Refractive Surgery-Vol 22, March 1996 at page 273
“After-Cataract: Studies of Chemical and Radiation Inhibition”, Roy et al, Contact and Intraocular Lens Medical Journal, Vol. 5, No. 4, October/December 1979, pp. 175-178

What is claimed:

1. An ophthalmic surgical instrument for polishing the capsule of an eye comprising:
a body having a top end and a bottom end, wherein the length of said body is at least five two times longer than the thickness of said body;
a tapered portion connected to the top end of the body, wherein said tapered portion is integrally joined to said body at an acute angle of from about 0.1 degrees to about 10 degrees; and
a biocompatible swab having a diameter from about 0.1 mm to about 5 mm attached to the tip of said tapered portion.

2. The instrument of claim 1, wherein said body is composed of a material selected from the group consisting of synthetic polymers, polycarbonate, polypropylene, polyethylene, polyurethane, plastic and metals.

3. The instrument of claim 1, wherein said biocompatible swab is composed of a material selected from the group consisting of nylon, silicone, acrylic compounds, teflon, polyesters, polyethylene, polyurethane and plastic.

4. The instrument of claim 1, wherein the total length of the instrument is between about 2 cm to about 18 cm and the length of the tapered portion is between about 0.1 cm to about 3 cm.

5. The instrument of claim 1, wherein the weight of the instrument is between 1 grams and 100 grams.

6. The instrument of claim 1, wherein the body is cylindrically shaped, triangularly shaped or rectangularly shaped.

7. An ophthalmic surgical instrument for polishing the capsule of an eye comprising: a ductile body having a top end and a bottom end, wherein the length of said body is at least two times longer than the thickness of said body;
a tapered portion connected to the top end of the body, wherein said tapered portion is integrally joined to said body at an acute angle between about 0.1 degrees to about 10 degrees; and
a biocompatible swab having a diameter between about 0.1 mm to about 5 mm attached to the tip of said tapered portion.

8. The instrument of claim 8, wherein the Young's modulus of said body and said tapered portion is between about 0.1 GPa to about 15 GPa.

9. The instrument of claim 8, wherein said body and said tapered portion is composed of a material selected from the group consisting of synthetic polymers, polycarbonate, polypropylene, polyethylene or polyurethane, plastic and metals.

10. The instrument of claim 8, wherein said biocompatible abrasive swab is composed of a material selected from the group consisting of nylon, silicone, acrylic compounds, teflon, polyesters, polyethylene, plastics or polyurethane.

11. The instrument of claim 8, wherein the total length of the instrument is between about 2 cm to about 18 cm and the length of the tapered portion is between about 0.1 cm to about 3 cm.

12. The instrument of claim 8, wherein the weight of the instrument is between 1 grams and 100 grams.

13. The instrument of claim 11, wherein the body is cylindrically shaped.

14. A method of preventing posterior capsular fibrosis or secondary opacification in an individual, comprising:
polishing epithelium cells from the capsule of an eye using the instrument of claim 1.

15. The method of claim 14, further comprising:
applying a liquid to said cells.

16. The method of claim 14, wherein application of said liquid is to clean said cells, to kill said cells or to inhibit the migration of said cells.

17. The method of claim 14, wherein said polishing cleans epithelial cells and cortical residual material so as to help prevent secondary opacification and posterior capsular fibrosis.

18. The method of claim 14, wherein said polishing cleans cortical residual material.

19. A method of preventing posterior capsular fibrosis or secondary opacification in an individual, comprising:
polishing epithelium cells from the capsule of an eye using the instrument of claim 7.

20. The method of claim 19, wherein said polishing cleans epithelial cells and cortical residual material so as to help prevent secondary opacification and posterior capsular fibrosis.

21. The method of claim 19, wherein said polishing cleans cortical residual material.

22. The method of claim 19, further comprising applying a liquid to said cells.

23. The method of claim 22, wherein application of said liquid is to clean said cells, to kill said cells or to inhibit the migration of said cells.
